(19) United States
(12) Patent Application Publication
(10) Pub. No.: US 2018/0344229 A1
(43) Pub. Date: Dec. 6, 2018

(54) PULSE OXIMETER SUGGESTS ANOTHER TEST

(71) Applicant: KONINKLIJKE PHILIPS N.V., Eindhoven (NL)

(72) Inventors: John CRONIN, BONITA SPRINGS, FL (US); Joseph George BODKIN, FORT MEYERS, FL (US)

(21) Appl. No.: 15/761,467

(22) PCT Filed: Sep. 22, 2016
(86) PCT No.: PCT/EP2016/072478

§ 371 Date: Mar. 20, 2018

Related U.S. Application Data
(60) Provisional application No. 62/222,482, filed on Sep. 23, 2015.

(30) Foreign Application Priority Data
Mar. 18, 2016 (EP) 16161205.6

(51) Int. Cl. A61B 5/1455; A61B 5/01; A61B 5/00; A61B 5/145; G16H 10/60; G16H 50/30

(52) U.S. Cl. CPC A61B 5/14552; A61B 5/01; A61B 5/746; A61B 5/7475; G16H 50/30; A61B 5/742; A61B 5/14546; G16H 10/60; A61B 5/0022

(57) ABSTRACT

A method for suggesting additional tests based on a patient's health status and medical records comprising: inputting via a user interface the patient's medical records; acquiring health parameters of the patient; determining the patient's health status based on the acquired health parameters; transmitting to a network server the determined patient's health status and the inputted patient's medical records to determine a health template based on the transmitted patient's health status and patient's medical records; receiving from the network server the determined health template; extracting from the received health template an oxygen saturation level threshold and a pulse rate threshold range; acquiring an oxygen saturation level and a pulse rate of the patient via a pulse oximeter; and suggesting additional tests from the acquired health template when the acquired oxygen saturation level and pulse rate are not within the extracted oxygen saturation level threshold and pulse rate threshold range.

### BACKGROUND OF THE INVENTION

Pulse Oximetry is an effective non-invasive method of measuring oxygen saturation (SpO₂) level and pulse rate of a patient. The measured SpO₂ level and pulse rate are good indicators for the health status of the patient. When the measured SpO₂ level and pulse rate do not fall within known normal ranges, these may indicate that the patient is experiencing an unknown condition. Additional testing may be done by a medical practitioner to the patient in order to determine the present condition of the patient. These additional tests may be specific to the patient's health status as well as patient's medical record. Thereby, there exist a need for a system that suggests additional tests based on a patient's health status and medical records.

### SUMMARY OF THE CLAIMED INVENTION

The systems and methods according to some embodiments of the present invention relates to systems and methods for suggesting additional tests based on a patient's health status and medical records including family history and pre-existing conditions. The system comprises a plurality of health sensors for acquiring health parameters of a patient, a pulse oximeter for acquiring an oxygen saturation level and a pulse rate of the patient, and a display device with a user interface for inputting the patient's medical records. The system further comprises a controller unit connected to the plurality of health sensors, the pulse oximeter, and the display device. The controller unit is further connected to a network server.

The method comprises inputting via a user interface the patient's medical records; acquiring health parameters of the patient via a plurality of health sensors; determining the patient's health status based on the acquired health parameters; transmitting to a network server the determined patient's health status and the inputted patient's medical records to determine via the network server a health template based on the transmitted patient's health status and patient's medical records; receiving from the network server the determined health template; extracting from the received health template an oxygen saturation level threshold and a pulse rate threshold range; acquiring an oxygen saturation level and a pulse rate of the patient via a pulse oximeter; and suggesting additional tests from the acquired health template when the acquired oxygen saturation level and pulse rate are not within the extracted oxygen saturation level threshold and pulse rate threshold range.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 illustrates a block diagram of a system for suggesting additional tests based on a patient's health status and medical records according to a preferred embodiment of the present invention.

FIG. 2 illustrates a flowchart according to a preferred embodiment of the present invention.

FIG. 3A illustrates of the display device.

FIG. 3B illustrates of the display device with the user interface.

FIGS. 4A and 4B illustrate a user interface according to a preferred embodiment of the present invention.

FIG. 5 illustrates a block diagram of a system for suggesting additional tests based on a patient's health status and medical records according to a second embodiment of the present invention.

FIG. 6 illustrates a flowchart of the pulse oximeter software.

FIG. 7 illustrates a flowchart of the network server software

FIG. 8 illustrates a network health template database.

### DETAILED DESCRIPTION OF THE EMBODIMENTS

The present invention relates to a method for suggesting additional tests based on a patient's health status and medical records comprising: inputting via a user interface the patient's medical records; acquiring health parameters of the patient via a plurality of health sensors; determining the patient's health status based on the acquired health parameters; transmitting to a network server the determined patient's health status and the inputted patient's medical records to determine via the network server a health template based on the transmitted patient's health status and patient's medical records; receiving from the network server the determined health template; extracting from the received health template an oxygen saturation level threshold and a pulse rate threshold range; acquiring an oxygen saturation level and a pulse rate of the patient via a pulse oximeter; and suggesting additional tests from the acquired health template when the acquired oxygen saturation level and pulse rate are not within the extracted oxygen saturation level threshold and pulse rate threshold range.

The present invention also relates to a system for suggesting additional tests based on a patient's health status and medical records comprising: a plurality of health sensors for acquiring health parameters of the patient; a pulse oximeter for acquiring an oxygen saturation level and a pulse rate of the patient; a display device with a user interface for inputting the patient's medical records; a controller unit connected to the plurality of health sensors, the pulse oximeter, and the display device; and a network server connected to the controller unit for determining a health template based on a patient's health status and the inputted patient's medical records.

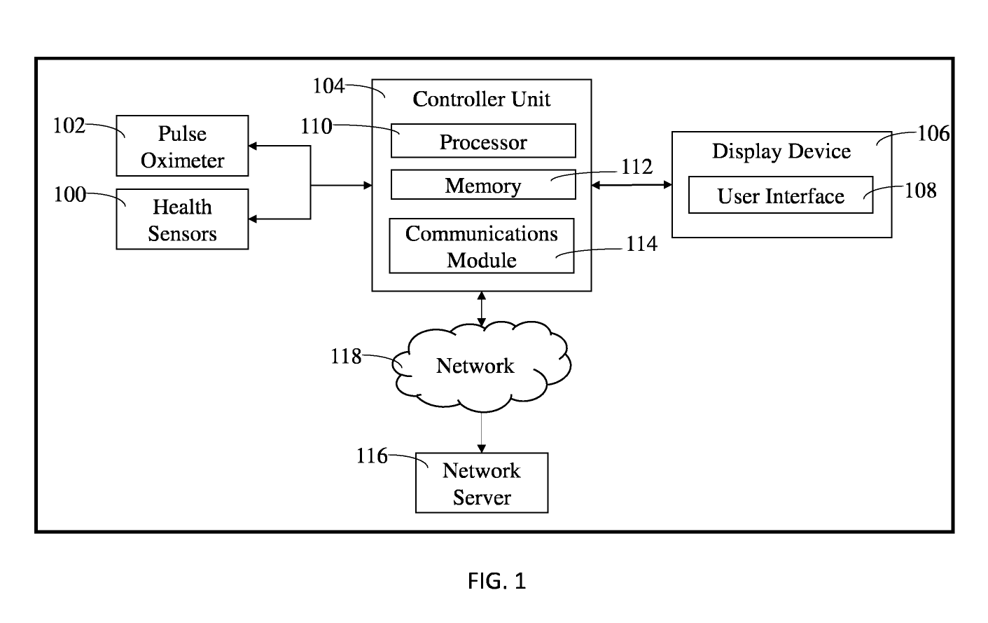

In a preferred embodiment of the present invention as illustrated in FIG. 1, a system for suggesting additional tests based on a patient's health status and medical records comprises a plurality of health sensors 100, a pulse oximeter 102, a controller unit 104, and a display device 106 with a user interface 108. The controller unit 104 comprises a processor pulse oximeter 102, a memory 112, and a communications module 114. Further, the plurality of health sensors 100, the pulse oximeter 102, and the display device 106 are connected to the controller unit 104. The controller unit 104 is further connected to a network server 116 via a communications link 118.

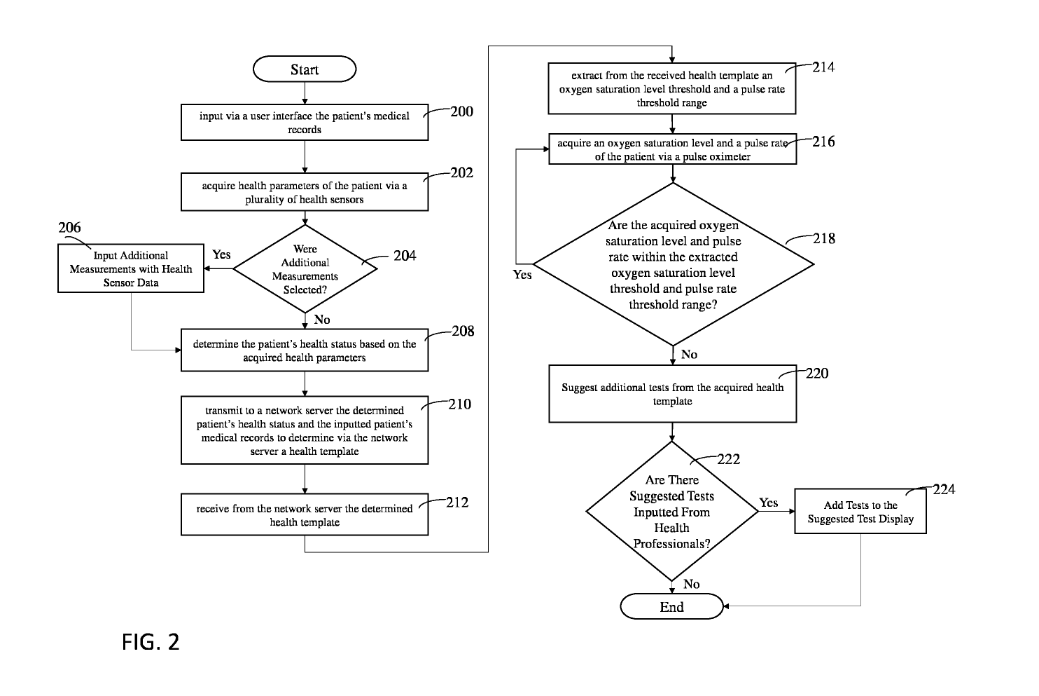

FIG. 2 illustrates a preferred method of the present invention. A medical practitioner inputs via a user interface 108 the patient's medical records (step 200). The inputted medical records may include the patient's disease, condition, current medications, family history, and various patient information such as age, weight, and height. Afterwards, the health parameters of the patient are acquired via the plurality of health sensors 100 (step 202). Examples of the health parameters acquired are immunoglobulin E (IgE) level and body temperature acquired by a blood testing device and a thermometer, respectively.

Based on the acquired health parameters (step 202), the health status of the patient is determined (step 208). Preferably the determination of the patient's health status, as described herein, is done automatically by the controller unit 104. Optionally, the determination of the patient's health status is done manually by the medical practitioner. The determined patient's health status may then be inputted in the user interface 108. The patient's health status and medical records may be stored in the memory 112 of the controller unit 104. Based on the acquired immunoglobulin E (IgE) level and body temperature, for example, the controller unit 104 determines the patient has an asthma and his body temperature is normal. Also the caregiver may perform tests in addition (204) to the health parameters that are acquired by the health sensors, such as respiratory rate, to allow for a more accurate health status of the patient (206).

Thereafter, the health status of the patient and the patient's medical records are transmitted to the network server 116 via the communications module 114. The transmission may be done wirelessly via Wi-Fi or wired via a USB cable. Then, the network server 116 determines a health template based on the transmitted patient's health status and medical records (step 210). In accordance with some embodiments of the invention, the health template may include an oxygen saturation level, a pulse rate threshold range, and additional tests relating to the patient's health status and medical records. The determined health template is then sent by the network server 116 to the controller unit 104 and is received by the controller unit 104 (step 212).

Next, the received health template is extracted by the controller unit 104 to extract the oxygen saturation level threshold, the pulse rate threshold range and additional tests relating to the patient's health status and medical records (step 214). These extracted threshold ranges are set by the controller unit 104 as triggers for suggesting additional tests. The setting of the triggers may be done automatically by the controller unit 104 or may be done manually by the medical practitioner via user interface 108. For example, the threshold range for pulse rate is less than 45 BPM while the threshold range for the oxygen saturation level is less than 94%.

After setting the extracted threshold ranges, the pulse oximeter 102 acquires an oxygen saturation level and a pulse rate of the patient (step 216). The controller unit 104 determines if the acquired oxygen saturation level and pulse rate are within the extracted oxygen saturation level threshold and pulse rate threshold range (step 218). If the acquired oxygen saturation level and pulse rate are within their respective threshold ranges, the pulse oximeter 102 continuously acquire the oxygen saturation level and the pulse rate of the patient (step 216). Alternatively, if the acquired oxygen saturation level and pulse rate are not within their respective threshold ranges, the controller unit 104 suggests additional tests extracted from the acquired health template (step 220). In addition, the caregiver may input their own suggested tests (222) and the caregiver suggested tests are added to the suggested test list on the user interface (224).

For example, the acquired oxygen saturation level and pulse rate, are 92% and 38 BPM, respectively. These acquired parameters are less than their respective threshold ranges. Following this, the controller unit 104 suggest additional tests extracted from the health template. Based on the patient's health status and medical records, the suggested additional tests are blood pressure testing and respiratory rate testing.

In accordance with the preferred embodiment of the invention, the suggested additional tests are displayed on the display device 106. An alarm may also be triggered via the display device 106 to alert the medical practitioner that suggested additional tests should be conducted on the patient.

The plurality of health sensors 100 are used to acquire health parameters of the patient. These acquired health parameters are used to determine the health status of the patient. Examples of the plurality of health sensors 100 that may be used include a thermometer, an ECG, an EKG, a blood sugar monitor and a blood pressure monitor. The pulse oximeter 102 of the present invention is preferably a portable pulse oximeter device adapted to be worn on a patient's finger and adapted to measure the oxygen saturation level and pulse rate of the patient. The network server 116 used herein, for example, the present may reside within the vicinity of the controller unit 104. In another embodiment of the present invention, the network server 116 may reside in a cloud server. The controller unit 104 is preferably a stand-alone device connected to a display device 106—which may be a laptop computer, a desktop computer, a mobile phone, a tablet computer, or a PDA. In another embodiment of the invention, the controller unit 104 may reside within the display device 106.

In accordance with the various embodiments of the present invention, the memory 112 may include high-speed random access memory or non-volatile memory such as magnetic disk storage devices, optical storage devices, or flash memory. Memory 112 may also store software instructions for facilitating processes, features and applications of the system disclosed in the invention. The communications module 114 may include any transmitter or receiver used for Wi-Fi, Bluetooth, infrared, NFC, radio frequency, cellular communication, visible light communication, Li-Fi, WiMax, ZigBee, fiber optic and other forms of wireless communication devices. Alternatively, the communications module 114 may include a physical channel such as a USB cable or other wired forms of communication.

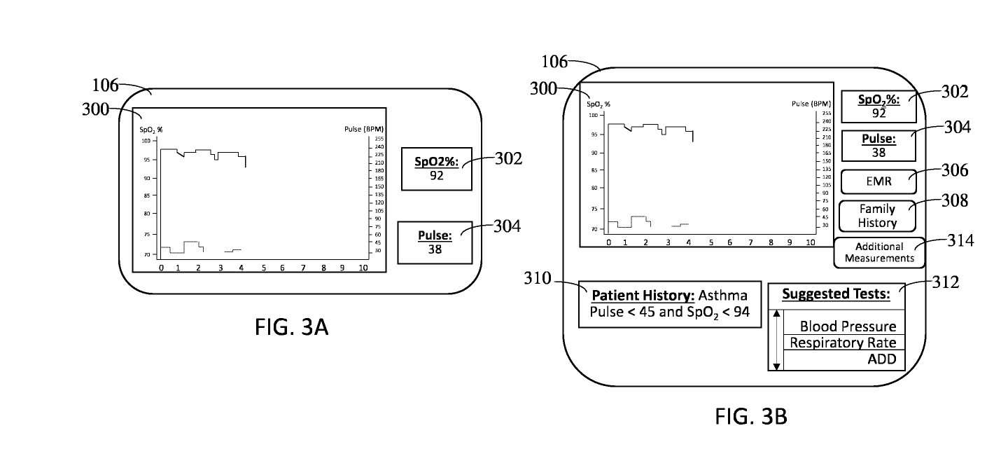

FIG. 3A illustrates an exemplary display device 106 known in the art. A display graph 300 shows a graph of oxygen saturation level readings and pulse rate readings versus time. This display graph 300 enables the medical practitioner to view the previous pulse oximeter 102 readings as well as the line trend of the readings. The current SpO₂ level 302 and pulse rate 304 readings are also displayed on the display device 106.

FIG. 3B illustrates an exemplary display device 106 with an exemplary user interface 108 in accordance to some of the embodiments of the system of the present invention. In some embodiments, the user interface 108 is integrated to the display device 106. The display device 106 is a touch-screen display capable of receiving tactile information from a user. The medical practitioner may input the patient's medical records and family history via Electronic Medical Records (EMR) Interface 306 and Family History Interface 308, respectively. These interfaces 306 and 308 will be discussed later. Additional measurements interface 314 allows the medical practitioner to look up additional medical measurements that are not displayed on the display and can select which measurements should be added to the patient's electronic medical records. Interface 310 shows the patient history which may be the current disease the patient has. Interface 310 also shows the extracted pulse rate threshold and oxygen saturation level threshold ranges from the health template. Suggested Tests Interface 312 shows the suggested additional tests relating to the patient's health status and medical records. As shown in the figure, the suggested additional tests for a patient with an asthma are blood pressure testing and respiratory rate testing. The medical practitioner is given the option to add a suggested test (optional suggested test) which will allow them to select their own tests that may seem relevant to perform the proper diagnoses on the patient, these selected tests will be added to the suggested test list.

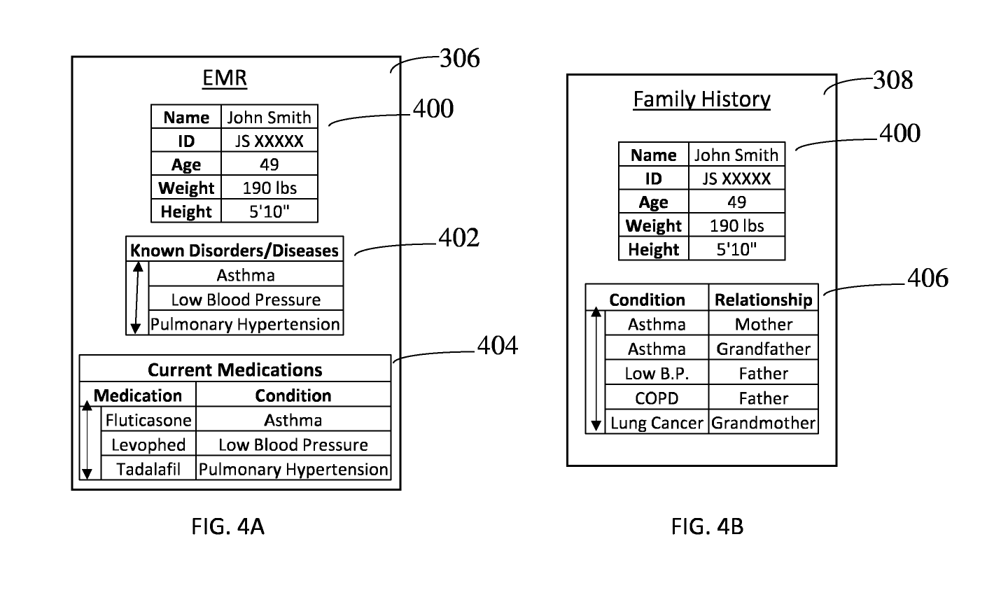

FIG. 4A illustrates an exemplary EMR Interface 306 in accordance with the preferred embodiment in the present invention. EMR Interface 306 displays various patient information 400, known disorders/diseases 402 of the patient, and current medications 404. FIG. 4B illustrates an exemplary Family History Interface 308 in accordance with the preferred embodiment in the present invention. Family History Interface 308 displays various patient information 400 and the patient's family members and their corresponding conditions 406.

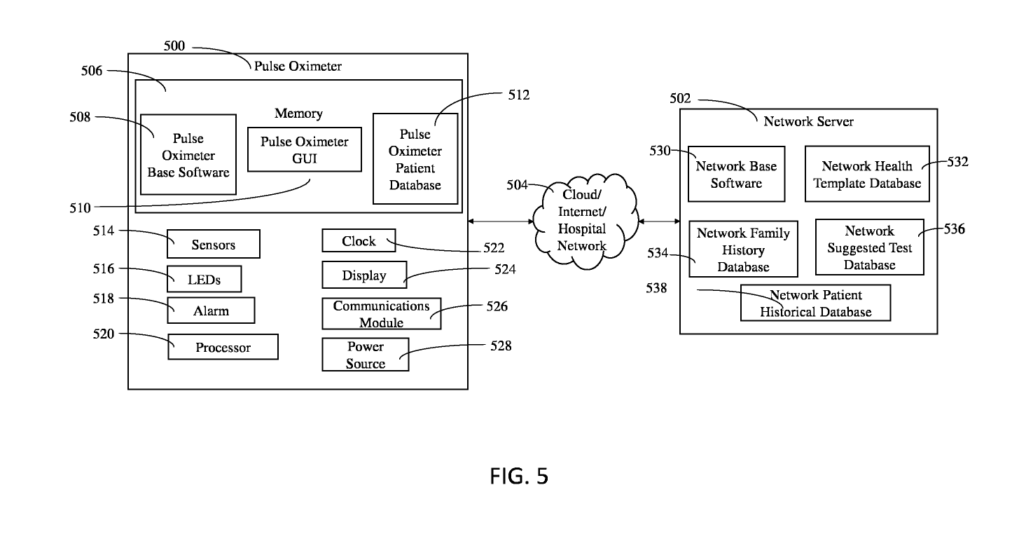

In a second embodiment of the present invention as illustrated in FIG. 5, a system for suggesting additional tests based on a patient's health status and medical records comprises a pulse oximeter 500 and a network server 502. The pulse oximeter 500 and the network server 502 are connected to each other via a communications link 504. The pulse oximeter 500 comprises a memory 506 for storing a pulse oximeter base software 508, a pulse oximeter GUI 510, and a pulse oximeter patient database 512. The pulse oximeter 500 further comprises a plurality of sensors 514, a plurality of LEDs 516, an alarm 518, a processor 520, a clock 522, a display 524, a communications module 526, and a power source 528. The network server 502 stores a network base software 530, a network health template database 532, a network family history database 534, a network suggested test database 536, and a network patient historical database 538.

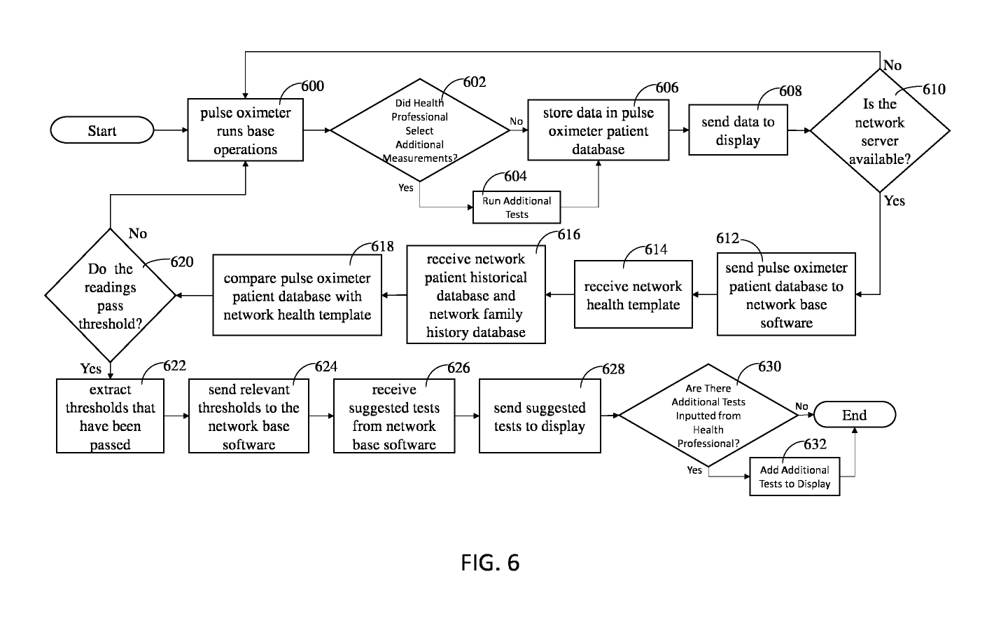

FIG. 6 illustrates the process of the pulse oximeter base software 508 described in the second embodiment of the present invention. The process starts with the pulse oximeter 500 running base operations such as taking oxygen saturation level and pulse rate readings (step 600). Then the pulse oximeter software determines if the medical practitioner selected additional measurements. If no additional tests were selected the pulse oximeter readings are stored in the pulse oximeter patient database (step 602). However, if they were selected, the additional tests are performed in order to collect the additional medical data (step 604). Then these readings are stored in the pulse oximeter patient database 512 (step 606) and are sent to display 524 (step 608). Afterwards, the pulse oximeter 500 checks if the network server 502 is available (step 610). If the network server 502 is not available, the pulse oximeter 500 continuously run the base operations (step 600). If the network server 502 is available, the data stored in the pulse oximeter patient database 512 are sent to the network base software 530 (step 612). Afterwards, the pulse oximeter 500 receives a network health template from the network server 502 (step 614).

Along with the network health template, the data stored in the network patient historical database 538 and network family history database 534 are also received by the pulse oximeter 500 (step 616). Next, the data stored in the pulse oximeter patient database 512 are compared to the corresponding data in the received network health template (step 618). Thereafter, the pulse oximeter 500 determines if the readings pass the threshold ranges (step 620). If readings did not pass the threshold ranges, the pulse oximeter 500 begins the process again (step 600). Alternatively, if the readings pass the threshold ranges, the pulse oximeter base software 508 extracts the thresholds that have been passed (step 622). Then, the extracted thresholds are sent to the network server 502 (step 624). Thereafter, the pulse oximeter 500 receives suggested tests from the network base software 530 (step 626). Finally, the received suggested tests sent to display 524 (step 628). The pulse oximeter software determines if there are any additional tests from the medical practitioner (step 630), if there are additional suggested tests the suggested tests are displayed on the suggested test list (step 632), and if there are no additional suggested tests, the process ends.

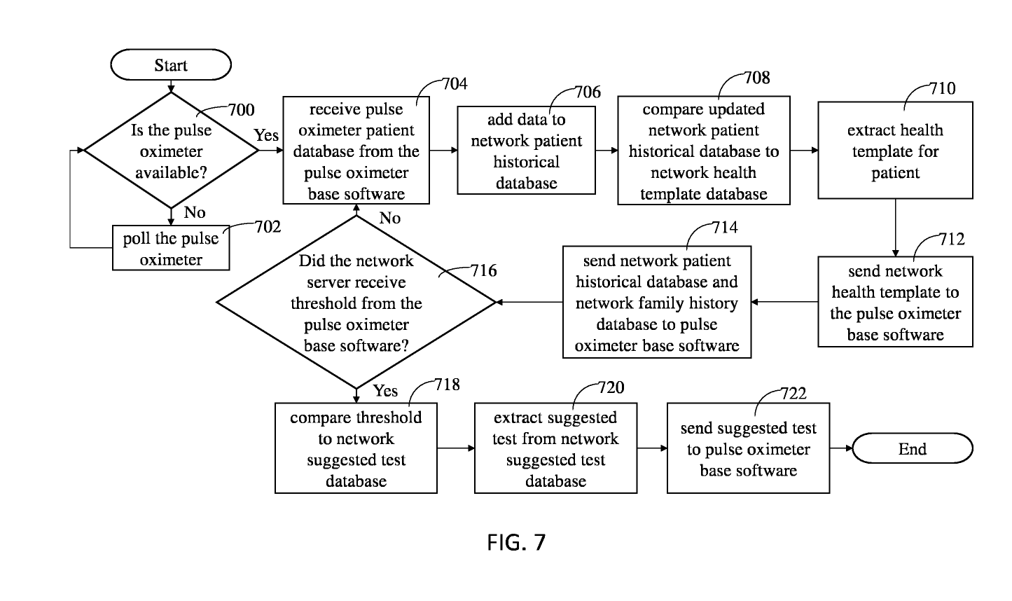

FIG. 7 illustrates the process of the network base software 530 described in the second embodiment of the present invention. The process starts at determining if the pulse oximeter 500 is available (step 700). If the pulse oximeter 500 is not available, the network server 502 continuously polls the pulse oximeter 500 (step 702). If the pulse oximeter 500 is available, the network server 502 receives the data stored in the pulse oximeter patient database 512 from the pulse oximeter base software 508 (step 704). The received data are then added to the network patient historical database 538 for updating (step 706). The network base software 530 compares the data stored in the updated network patient historical database 538 with the corresponding data stored in the network health template database 532 (step 708). Afterwards, the health template is extracted (step 710) and then sent to the pulse oximeter base software 508 (step 712) along with the data stored in the network patient historical database 538 and the network family history database 534 (step 714). Then, the network base software 530 checks if the network server 502 received the extracted threshold from the pulse oximeter base software 508 (step 716). If the network server 502 did not receive the extracted threshold, the network base software 530 returns to the process of receiving the data stored in the pulse oximeter patient database 512 from the pulse oximeter base software 508 (step 704). Alternatively, if the network server 502 did not received the extracted threshold, the network base software 530 compares the received threshold to corresponding data stored in the network suggested test database 536 (step 718). Finally, the suggested test is extracted from the network suggested test database 536 (step 720) and then sent to the pulse oximeter base software 508 (step 722).

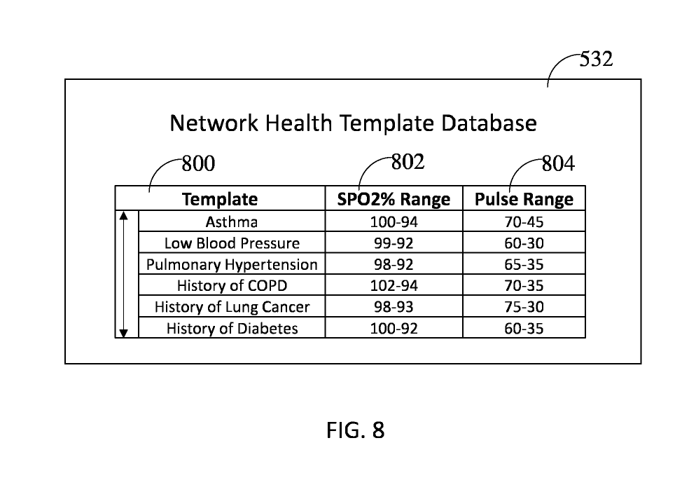

FIG. 8 displays an exemplary Network Health Template Database (532), which displays the various health templates (800) that the health status is compared to and the corresponding SPO2 (802) and pulse thresholds for each of the health templates (804).

The present invention is not intended to be restricted to the several exemplary embodiments of the invention described above. Other variations that may be envisioned by those skilled in the art are intended to fall within the disclosure.

1. A method for suggesting additional tests based on a patient's health status and medical records, the method comprising:
inputting via a user interface the patient's medical records;
acquiring health parameters of the patient via a plurality of health sensors;
determining the patient's health status based on the acquired health parameters;
transmitting to a network server the determined patient's health status and the inputted patient's medical records to determine via the network server a health template corresponding to the transmitted patient's health status and medical records;
receiving from the network server the determined health template; extracting from the received health template an oxygen saturation level threshold range and a pulse rate threshold range;
acquiring an oxygen saturation level and a pulse rate of the patient via a pulse oximeter; and
suggesting one or more additional different test based on the acquired health template when the acquired oxygen saturation level and pulse rate are not within the extracted oxygen saturation level threshold range and pulse rate threshold range, respectively,
wherein the additional test is selected from a blood pressure test, a respiratory rate test, and an optional suggested test.

2. The method of claim 1, wherein the health template is selected from Asthma, low blood pressure, pulmonary hypertension, history of COPD, history of lung cancer, and history of diabetes.

3. The method of claim 2, wherein the health template is Asthma, the oxygen saturation level threshold range is 100-94 SpO2%, and the pulse rate threshold range is 70-45 BPM.

4. The method of claim 1, wherein the additional test is selected from a blood pressure test, a respiratory rate test, and an optional suggested test.

5. The method of claim 1, wherein the medical records includes the patient's disease, condition, current medications, family history, patient information including age, weight, height, or any combination thereof.

6. (canceled)

7. The method of claim 1, wherein the acquired health parameters include immunoglobulin E (IgE) level, body temperature, or both.

8. The method of claim 1, wherein determining the patient's health status is done automatically by the controller unit.

9. The method of claim 1, further comprising storing the patient's health status and medical records in a memory of the controller unit.

10. The method of claim 1, further comprising acquiring the health parameters of the patient for determining the health status of the patient.

11. The method of claim 1, wherein the additional test is extracted from the acquired health template.

12. The method of claim 1, wherein acquiring the oxygen saturation level and the pulse rate of the patient is done continuously when the acquired oxygen saturation level and pulse rate are within their respective threshold ranges.

13. (canceled)

14. (canceled)

15. The method of claim 1, further comprising triggering an alarm via the display device to alert a medical practitioner that the suggested additional tests should be conducted on the patient.

16. (canceled)

17. A system for suggesting additional tests based on a patient's health status and medical records using the method of claim 1, the system comprising:
a plurality of health sensors for acquiring health parameters of the patient; a pulse oximeter for acquiring an oxygen saturation level and a pulse rate of the patient;
a display device with a user interface for inputting the patient's medical records;
a controller unit connected to the plurality of health sensors, the pulse oximeter, and the display device; and
a network server connected to the controller unit for determining a health template based on the patient's health parameters and the inputted patient's medical records.

18. The system of claim 17, wherein the network server resides within a cloud.

19. The system of claim 17, wherein one of:
the controller unit is a stand-alone device connected to the display device;
the controller unit resides within the display device; and
the controller unit resides within the pulse oximeter

20. (canceled)

21. The system of claim 17, wherein the user interface is integrated to the display device.

22. The system of claim 17, wherein the user interface includes an additional measurements interface.

23. (canceled)
